(19) United States
(12) Patent Application Publication
(10) Pub. No.: US 2026/0237499 A1
(43) Pub. Date: Aug. 13, 2026

(54) ARTIFICIAL INTELLIGENCE AGENT FOR OPERATING ROOMS AND SURGERY

(71) Applicant: Alcon Inc., Fribourg (CH)

(72) Inventors: Zhuoran Wu, Rancho Cucamonga, CA (US); Lu Yin, Keller, TX (US); Vignesh Suresh, Woodland Hills, CA (US); Ramesh Sarangapani, Coppell, TX (US); Joseph Weatherbee, San Clemente, CA (US)

(73) Assignee: ALCON CORPORATION, (omitted)

(21) Appl. No.: 19/529,774

(22) Filed: Feb. 4, 2026

Related U.S. Application Data
(60) Provisional application No. 63/756,537, filed on Feb. 10, 2025.

(51) Int. Cl. G16H 40/20; G16H 50/20

(52) U.S. Cl. CPC G16H 40/20; G16H 50/20

(57) ABSTRACT

A computer-implemented method of machine learning based medical treatment optimization. Embodiments include receiving, by an artificial intelligence (AI) agent, a request related to treating a patient. Embodiments include retrieving medical data that is related to the request from one or more source devices, whereint he medical data includes multiple data modalities. Embodiments include generating, using a multimodal machine learning model, response content related to treating the patient based on the request and the medical data. Embodiments include providing the response content via an output device.

### CROSS-REFERENCE TO RELATED APPLICATIONS

This application claims priority to and benefit of U.S. Provisional Patent Application No. 63/756,537, filed February 10, 2025, which is incorporated by reference herein in its entirety, and is hereby expressly made a part of this specification.

### INTRODUCTION

In a typical operating room or other medical treatment context, there is a vast amount of data being generated and utilized. This may include information from medical equipment, human communication, pre-operative data, human movement, and the like.

Leveraging such data in order to automatically generate useful recommendations or other content for use by medical professionals in connection with the treatment of patients can be challenging. For example, given the varying types, modalities, formats, and other attributes of such data, it is difficult to automatically analyze such data in a relational or holistic manner. Automatically synthesizing data across different modalities (e.g., text, images, video, audio, and/or the like), that relates to different purposes, that can be stored in different formats, and/or the like is technically difficult. Given these technical challenges, automated recommendations or other content generated based on such data using existing techniques may be inaccurate, may not be contextually informed, and/or otherwise may have limited utility.

Accordingly, there is a need for improved techniques for automated analysis and content generation based on disparate data sources related to the medical treatment of patients.

### SUMMARY

In certain embodiments, one general aspect includes a computer-implemented method for machine learning based medical treatment optimization. The computer-implemented method includes: receiving, by an artificial intelligence (AI) agent, a request related to treating a patient; retrieving medical data that is related to the request from one or more source devices, wherein the medical data includes multiple data modalities; generating, using a multimodal machine learning model, response content related to treating the patient based on the request and the medical data; and providing the response content via an output device

In certain embodiments, another general aspect includes a system. The system includes a memory having executable instructions and a processor in communication with the memory. The processor is configured to execute the instructions to perform the computer-implemented method for machine learning based medical treatment optimization described above.

In certain embodiments, another general aspect includes a computer-program product including a non-transitory computer-usable medium having computer-readable program code embodied therein. The computer-readable program code is adapted to be executed to implement the computer-implemented method for machine learning based medical treatment optimization described above.

### BRIEF DESCRIPTION OF THE DRAWINGS

FIG. 1 illustrates an example of computing components related to machine learning based medical treatment optimization, in accordance with certain embodiments of the present disclosure.

FIG. 2 illustrates an example process related to training a machine learning model for machine learning based medical treatment optimization, in accordance with certain embodiments of the present disclosure.

FIG. 3 illustrates an example of an artificial intelligence agent for machine learning based medical treatment optimization, in accordance with certain embodiments of the present disclosure.

FIG. 4 illustrates an example related to machine learning based medical treatment optimization, in accordance with certain embodiments of the present disclosure.

FIG. 5 illustrates another example related to machine learning based medical treatment optimization, in accordance with certain embodiments of the present disclosure.

FIG. 6 illustrates an example of a process related to machine learning based medical treatment optimization, in accordance with certain embodiments of the present disclosure.

FIG. 7 illustrates an example of a computing device for machine learning based medical treatment optimization, in accordance with certain embodiments of the present disclosure.

### DETAILED DESCRIPTION

Large amounts of data are generated and stored in connection with the treatment of patients in medical contexts, such as in connection with surgeries and other procedures. This data may be captured and stored by a variety of different devices, in different formats, for different purposes, and in different modalities such as text, images, video, audio, and the like.

Aspects of the present disclosure enable the use of such disparate types of data for automated analysis and generation of accurate content such as recommendations related to patient medical care using one or more artificial intelligence (AI) agents. According to certain aspects, an AI agent may utilize a multimodal machine learning model such as a multimodal large language model (MLLM) to automatically generate outputs such as recommendations or other types of content to provide to medical professionals based on various types of input medical data (e.g., having different modalities).

Multimodal machine learning models such as MLLMs transcend traditional text-based interfaces and provide the ability to comprehend and generate content across a wide array of formats, including text, images, audio, and video. A multimodal machine learning model canintegrate and interpret diverse forms of data, offering an unprecedented level of contextual understanding and interaction. For example, an AI agent may utilize such a model to perceive its environment based on various types of data in multiple modalities, thereby maximizing its chances of achieving its goals. In the context of a medical operating room, an AI agent can serve as a central hub, processing a wide array of data to facilitate efficient and effective surgical procedures.

In a typical operating room, there is a vast amount of data being generated and utilized. This may include information from medical equipment, human communication, pre-operative data, human movement, and/or the like. An AI agent, according to techniques described herein, can process all these types of data in real-time, providing valuable insights and assistance to the medical team, upon request or otherwise. For example, medical equipment such as monitors, ventilators, and surgical instruments generate a wealth of data. An AI agent can monitor these data streams, alerting a medical team to any anomalies and helping to ensure a patient’s safety.

Effective communication is vital in a surgical setting. An AI agent, according to aspects of the present disclosure, can analyze spoken language, gestures, and other forms of communication and generate applicable outputs to facilitate better teamwork and prevent misunderstandings. Pre-operative data such as medical information (e.g., which may include medical history and other medical data), lab results, and imaging studies are crucial for planning and executing a successful surgery. An AI agent can analyze this data, helping the surgical team to make informed decisions. An AI agent can also monitor the movements of the surgical team, helping to optimize workflows and prevent accidents. For instance, an AI agent can utilize a multimodal machine learning model to process various types of input (e.g., text, speech, images, etc.) and generate various types of output (e.g., alerts, recommendations, reports, etc.). This ability allows the AI agent to adapt to the unique needs and workflows of each surgical team.

AI agents described herein can assist in various aspects of medical care, includingsurgical planning, intraoperative assistance, postoperative care, postoperative assistance, and the like. By analyzing pre-operative data, an AI agent can help a surgical team plan the most effective approach. During surgery, an AI agent can monitor data from medical equipment and alert the team to any potential issues. After surgery, an AI agent can assist with patient monitoring and follow-up care. AI agents described hereinhave the potential to revolutionize the way surgeries are performed, making them safer, more efficient, and more effective. By serving as a central hub for data processing in the operating room, an AI agent can provide invaluable assistance to the surgical team and ultimately improve patient outcomes.

In some aspects, a multimodal machine learning model utilized by an AI agent has been fine-tuned based on domain-specific data such as medical care information, surgery information, surgery steps, video annotations, surgery records, patient information, operating room inventory, stock information, sensor data, and/or the like. An AI agent may deploy in an operating room, in a server room of a clinical facility, and/or the like, and may be connected to medical input and output devices as well as other digital devices. In certain aspects, a main AI agent is deployed into an AI agent computer that is placed in an operating room or other location associated with a clinical facility, and smaller “local” AI agents are deployed to other medical devices, such as running on associated medical device computers. Furthermore, the main AI agent may connect to remote cloud-based computing resources that enable performing of actions that utilize larger amount of computing resources. Various actions may be performed using the local AI agent at a medical device and/or the main AI agent associated with the clinical facility when appropriate in order to maximize efficiency and data security, while remote resources may be used as appropriate in a secure manner based on resource requirements.

Embodiments of the present disclosure accomplish various technical improvements. For example, utilizing multimodal machine learning techniques described herein to automatically generate content such as recommendations related to clinical treatment of patients based on various types of data overcomes technical challenges associated with automated analysis of such data by enabling relationships among such data to be automatically identified and used in content generation despite the varying modalities, formats, and types of such data. Deploying an AI agent described herein in a clinical setting, such as in an operating room enables live, interactive assistance to be automatically provided to medical professionals in connection with the treatment of patients based on information captured by various medical devices, sensors, and/or the like, such as allowing for automatically generating responses to natural language requests with a higher level of accuracy and utility than would be possible with existing techniques. In some cases, a user of systems implementing techniques described herein may be enabled to request the generation or modification of content in particular modalities (e.g., text, image, video, audio, sensor data, and/or the like) using intuitive natural language queries, and such content may be automatically generated in an accurate manner based on a variety of different underlying data sources of one or more modalities.

Certain aspects of the present disclosure provide resource-efficient and secure automated processing of medical data related to patients by dynamically selecting AI agents on different devices (e.g., a central system, individual medical devices, cloud resources, and/or the like) for performing certain tasks, such as based on the tasks to be performed, resource requirements of such tasks, security requirements of such tasks, and/or the like.

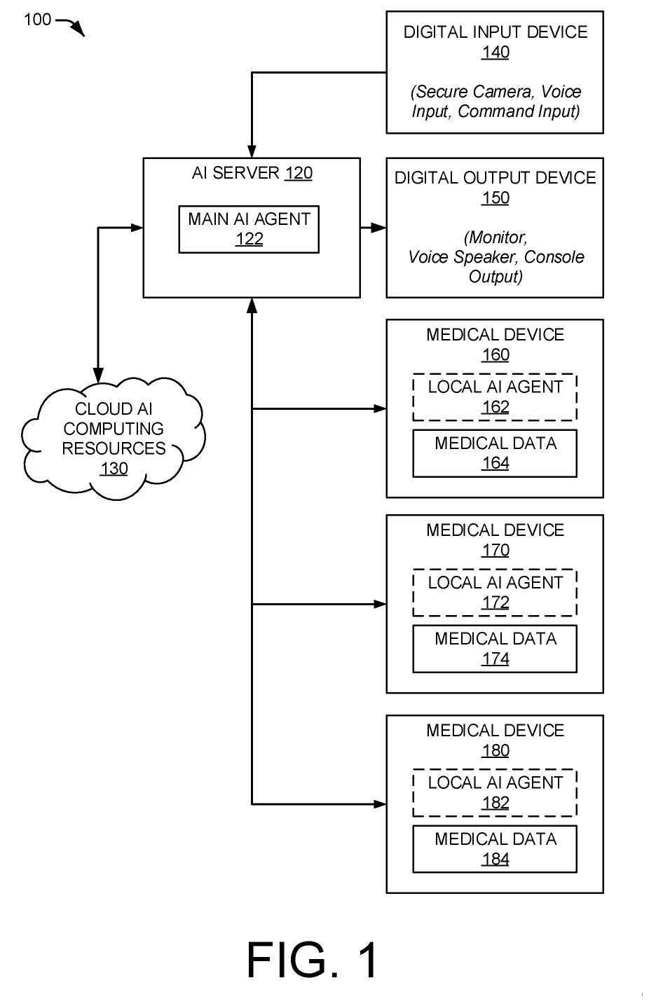

FIG. 1 illustrates an example computing environment 100 comprising computing components related to machine learning based medical treatment optimization, in accordance with certain embodiments of the present disclosure.

In computing environment 100, an artificial intelligence (AI) server 120 running a main AI agent 122 is connected to a plurality of devices such as a digital input device 140, a digital output device 150, and medical devices 160, 170, and 180. AI server 120 is also connected to cloud AI computing resources 130. For example, AI server 120 may be connected to the plurality of devices via a network such as a wireless or wired connection (e.g., any type of connection over which data may be transmitted) and may be connected to cloud computing resources 130 over a network such as the Internet or another connection over which data may be transmitted.

AI server 120 may be located in a clinical facility. In some aspects, AI server 120 is a physical or virtual computing device that runs main AI agent 122 and utilizes digital input device 140 and digital output device 150 to receive requests from one or more users and provide requested content in response to such requests, such as based on using a multimodal machine learning model to automatically generate the requested content based on data from medical devices 160, 170, and/or 180. Digital input device 140 may include, for example, one or more devices such as a camera, microphone, mouse, keyboard, touch screen, and/or the like that enable receiving visual input (e.g., images and/or video), audio input, touch input, click input, text input, and/or the like. In some cases, certain inputs received via digital input device 140 and/or from one or more other sensors associated with one or more other devices may be referred to as sensor data (e.g., which may include input from the user and/or other data about a user that is captured via one or more sensors and/or input devices). Digital output device 150 may include, for example, one or more devices such as a monitor or other screen, speaker, and/or the like for providing outputs in the form of text, images, video, sound, and/or the like.

Each of medical devices 160, 170, and 180 may be representative of a device capable of capturing, generating, and/or storing data related to clinical treatment of a patient. For example, medical devices 160, 170, and 180 may include one or more ventilators, surgical instruments, health monitoring devices, activity monitoring devices, cameras, sensors, medical data storage components, and/or the like. Each of medical devices 160, 170, and 180 may include a respective local AI agent 162, 172, or 182, which may utilize one or more machine learning models (e.g., a multimodal machine learning model) to perform automated analysis of medical data 164, 174, or 184. Medical data 164, 174, and 184 from medical devices 160, 170, and 180 may be provided to AI server 120 for automated analysis by main AI agent 122. For example, medical data 164, 174, and 184 may be provided to AI server 120 at regular intervals, upon request from AI server 120, when one or more other conditions occur, and/or the like.

Local AI agents 162, 172, and 182 may perform certain automated analysis that can be completed efficiently using the local resources of medical devices 160, 170, and 180. For example, certain types of anomaly detection, featurization, and event generation operations that require minimal amounts of computing resources may be performed by local AI agents 162, 172, and 182, and outputs from these operations may be provided to AI server 120 as appropriate, such as for further analysis and/or processing. In one example, an alert indicating a detected anomaly in one of medical data 164, 174, or 184 from one of local AI agents 162, 172, or 182 is provided to AI server 120 and AI server 120 provides such an alert for display or other form of output via digital output device 150.

Cloud AI computing resources may comprise one or more computing devices that are remote from a facility associated with AI server 120, digital input device 140, digital output device 150, and medical devices 160, 170, and 180. For example, cloud AI computing resources 130 may comprise one or more cloud servers that may be utilized by main AI agent 122 under certain conditions, such as to perform operations that are resource intensive (e.g., operations for which an expected amount of computing resource utilization is above a threshold and/or operations of certain types). In some aspects AI agent(s) utilize all data in-house and share data only as necessary with remote cloud resources (e.g., when additional computing resources are needed, when a client requests cloud processing, and/or the like) in order to improve security and data privacy.

Main AI agent 122 may perform automated analysis of data (e.g., medical data 164, 174, and/or 184), such as based on one or more requests received via digital input device 140 and/or without such a request, in order to generate content such as recommendations related to clinical treatment of a patient. In one example, a medical professional provides a request via digital input device 140 (e.g., via text, voice, video, and/or the like) for information regarding a next step of a surgical procedure, and main AI agent 122 retrieves data related to the request. For example, the data may include a subset of medical data 164, 174, and/or 184 that relates to a patient and/or procedure associated with the request. The data that is retrieved may include, for example, medical information (e.g., which may include medical history data and other medical data), measured health data, movement information, data about a procedure or medical condition, patient attributes, and/or the like. Patient attributes may include information about a patient, such as personal characteristics (e.g., age, gender, and/or the like), medical information (e.g., known medical conditions, information about the extent of known medical conditions, procedures that have been performed on the patient, medications taken by the patient, information about medical conditions of family members, and/or the like), and/or other information about the patient and/or the patient’s medical condition. Main AI agent 122 may be a software component that performs operations related to automated generation of content, such as retrieving/receiving relevant data, providing the relevant data along with a prompt to a multimodal machine learning model, and receiving an output from the multimodal machine learning model in response.

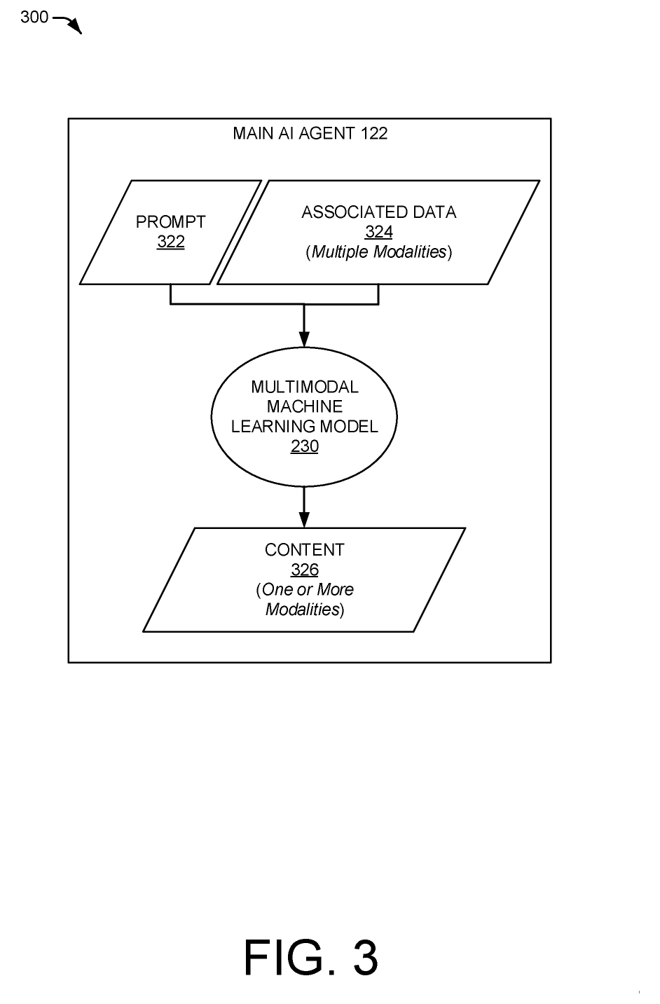

For instance, main AI agent 122 may provide the retrieved data that is related to the request to the multimodal machine learning model along with a prompt that is based on the request, as described in more detail below with respect to FIG. 3. The prompt may, for example, be a natural language prompt instructing the multimodal machine learning model to generate a recommendation related to clinical treatment of the patient according to the request. In some cases the request itself is used as a prompt, while in other cases a prompt may be generated based on the request, such as automatically populating a prompt template based on the request, using a language processing machine learning model to automatically generate the prompt based on the request, using rules to automatically generate the prompt based on the request, and/or the like. The request and the prompt may specify a modality for the output, such as text, audio, image, video, and/or the like, and multimodal machine learning model may generate the output in the specified modality accordingly. For instance, the request and prompt may specify that the requested information regarding a next step of a surgical procedure is to be output in the form of text and/or an image depicting the next step or a surgical instrument or item related to the next step. The multimodal machine learning model may include a language processing machine learning model, one or more diffusion models capable of analyzing and/or generating audio, video, and/or image content, and/or the like. Thus, the multimodal machine learning model may be capable of analyzing and/or generating content in such various modalities.

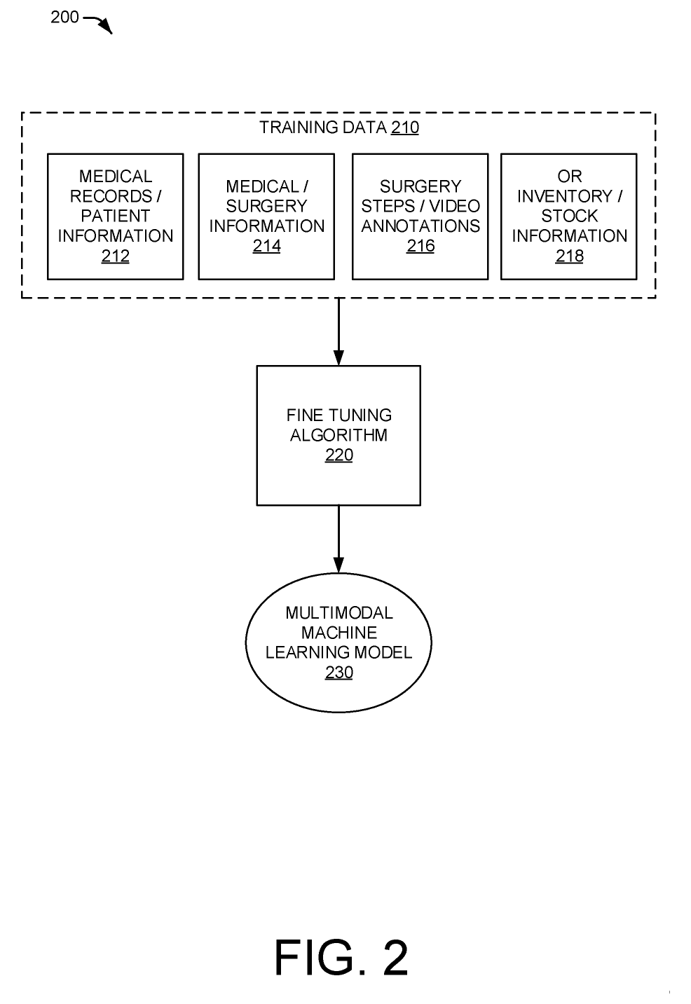

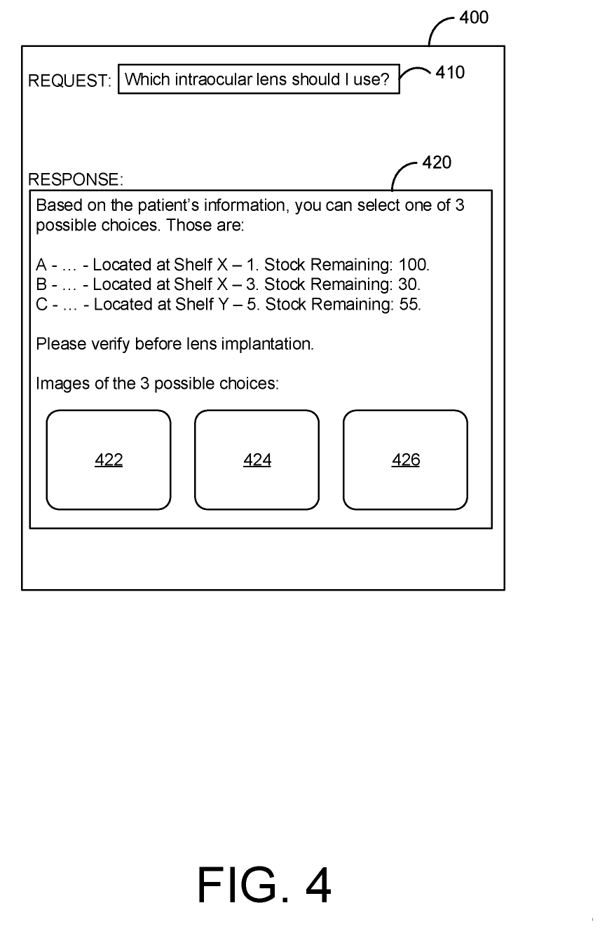

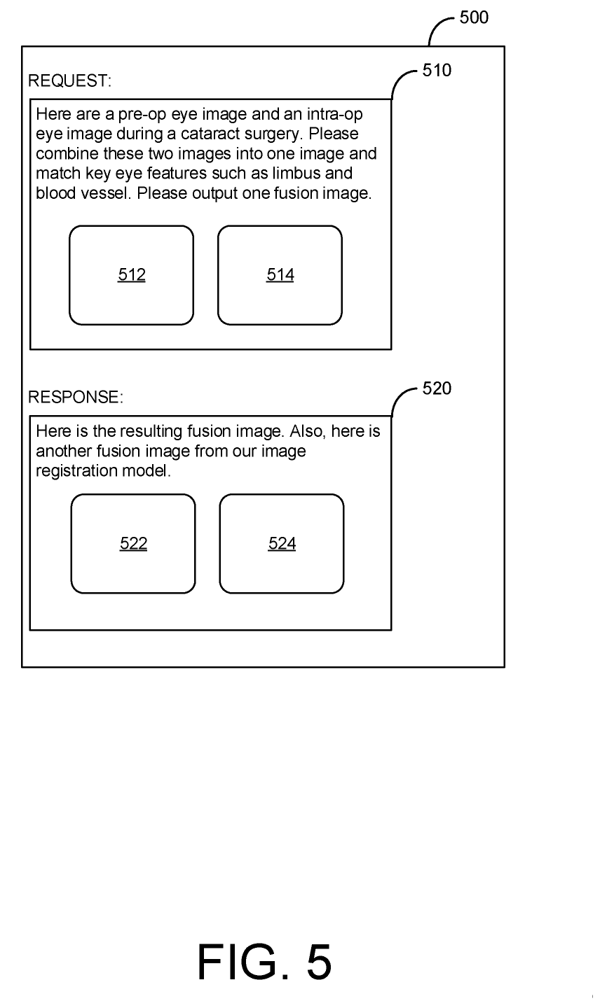

As described in more detail below with respect to FIG. 2, the multimodal machine learning model may have been fine-tuned based on data specific to a domain in which it used, such as clinical treatment of patients. For example, the multimodal machine learning model may have been fine-tuned based on historical medical data (e.g., from medical devices 160, 170, and/or 180) and/or other data to analyze and generate content based on such data. Certain examples of utilizing main AI agent 122 to generate content in response to requests are explained in more detail below with respect to FIGS. 4 and 5. Similar techniques may also be employed in connection with using local AI agents 162, 172, and/or 182 to generate content based on medical data and/or using cloud computing resources 130 (e.g., by main AI agent 122) to generate content based on medical data (e.g., by running a multimodal machine learning model on cloud AI computing resources 130).

In some aspects, anomaly detection may be performed by main AI agent 122 and/or one or more of local AI agents 162, 172, and/or 182 based on various types of data. For example, an AI agent may receive medical data captured, generated, and/or stored by one or more medical devices, and may process such data using a multimodal machine learning model and/or one or more rules to determine whether an anomalous condition is present. For example, such an AI agent may determine a baseline value or range for one or more types of data based on historical values for those types of data that are known to be associated with a normal or stable condition, and may determine an anomaly based on detecting a deviation from such a baseline, such as by more than a threshold amount. A machine learning model may be trained or fine-tuned for such anomaly detection based on historical data associated with labels indicating whether the data is or is not anomalous. In some cases, unsupervised learning may be used to identify baseline(s) for one or more types of data, and deviation from such baseline(s) by more than a threshold amount may be identified as an anomaly.

Content generated by main AI agent 122 and/or local agent 162, 172, and/or 182, such as content generated in response to a request, a notification generated based on an anomaly, and/or the like may be provided via digital output device 150. For example, text, image, video, and/or audio content may be output via digital output device 150 to one or more medical professionals.

FIG. 2 illustrates an example 200 related to training a multimodal machine learning model for machine learning based medical treatment optimization, in accordance with certain embodiments of the present disclosure. Example 200 includes a multimodal machine learning model 230, which may be utilized by one or more of main AI agent 122 and/or local agents 162, 172, and/or 182 of FIG. 1.

In example 200, training data 210 is used by a fine-tuning algorithm 220 to train or fine tune multimodal machine learning model 230. Training data 210 includes (or is based on) medical records / patient information 212, medical / surgery information 214, surgery steps / video annotations 216, operating room (OR) inventory / stock information 218, and/or the like. Medical records / patient information 212 generally include data about medical history and/or personal attributes of one or more patients, such as personal characteristics (e.g., age, gender, and/or the like), medical information (e.g., known medical conditions, information about the extent of known medical conditions, procedures that have been performed on the patient, medications taken by the patient, information about medical conditions of family members, and/or the like), and/or other information about the patient and/or the patient’s medical condition. Medical / surgery information 214 may include information about medical procedures, treatments, surgeries, and/or the like, such as being captured by one or more medical devices during performance of such procedures, treatments, surgeries, and/or the like, and/or records of such activities, and/or data describing proper performance of and/or outcomes of such procedures, treatments, surgeries, and/or the like. Surgery steps / video annotations 216 may include information about steps that are performed during surgeries, such as descriptions of such steps, images of such steps, video of such steps, annotations associated with video and/or images of such steps, audio of such steps, information about items (e.g., surgical instruments and/or other related items) associated with performing such steps (e.g., text, images, video, and/or audio related to such items), and/or the like. In some aspects, surgery steps / video annotations 216 may include information about the ordering of steps that are to be performed in surgeries and/or how to respond to issues, anomalies, and/or other events related to such steps. OR inventory / stock information 218 may include, for example, information about the numbers, types, locations, descriptions, and/or the like of various items in the inventory of a clinical facility, such as indicating which items are in stock, where such items are located, pictures of such items, textual descriptions of such items, information about the procedures, treatments, surgeries, and/or the like in which such items may be used, unique identifiers of such items, and/or the like. It is noted that the types of data depicted and described with respect to training data 210 are included as examples, and other types of data may also be included in training data 210.

Fine tuning algorithm 220 generally utilizes training data 210 to train multimodal machine learning model 230. For example, training algorithm 220 may involve supervised and/or unsupervised learning techniques by which multimodal machine learning model 230 is trained based on training data 210.

In some embodiments, labeled training data such as including sets of input features (e.g., prompts and subsets of medical records / patient information 212, medical / surgery information 214, surgery steps / video annotations 216, operating room (OR) inventory / stock information 218, and/or the like) labeled with manually generated and/or manually validated content generated based on such input features is used in a supervised learning process to train machine learning model 120. In a typical supervised learning process, a set of training inputs is provided to a model, the model generates an output in response to the set of training inputs, the generated output is compared to a label associated with the training inputs, and one or more parameters of the model are adjusted based on the comparing, such as iteratively until one or more conditions are met. For instance, the one or more conditions may relate to an objective function (e.g., a cost function), or may relate to whether the outputs produced by the model based on the training inputs match the labels associated with the training inputs or whether a measure of error between training iterations is not decreasing or not decreasing more than a threshold amount. The conditions may also include whether a training iteration limit has been reached. Parameters adjusted during training may include, for example, hyperparameters, values related to numbers of iterations, weights, functions used by nodes to calculate scores, and the like. In some embodiments, validation and testing are also performed for a machine learning model, such as based on validation data and test data, as is known in the art.

The training processes described above are included as examples, and other methods of training multimodal machine learning model 230 based on training data 210 are possible. In some embodiments, fine tuning algorithm 220 may involve one or more unsupervised learning processes (e.g., clustering), semi-supervised learning processes, and/or supervised learning processes. For example, unsupervised learning techniques or semi-supervised learning techniques may be used to analyze data and identify patterns. The results of such unsupervised and/or semi-supervised learning techniques may then be used in a supervised learning process, such as labeling input features for use in supervised learning based on such results. In other embodiments, labeled training data for a supervised learning process may be generated based on manual analysis of data and/or based on manual confirmation of results of an unsupervised learning process.

It is understood that a variety of machine learning techniques exist for such a training process, and any suitable machine learning algorithm(s) and/or model(s) may be used to train and/or fine tune multimodal machine learning model 230.

Multimodal machine learning model 130 may have been trained in advance of being fine-tuned. For example, such pre-training may have been based on a large training data set that is more general in scope than training data 210, such as not being limited to a domain associated with training data 210. Techniques for training a multimodal machine learning model are known in the art.

Multimodal machine learning model may, for example, a multimodal large language model (MLLM), and may include multiple models that are configured to analyze and/or generate different modalities. For example, multimodal machine learning model 230 may include an image input encoder, an audio input encoder, and a video input encoder that generate embeddings of image, audio, and video data, respectively. Such encoders may be used to convert different types of input data into embeddings that can be processed by a large language model (LLM) within multimodal machine learning model 230, such as along with text data that can also be processed in embedding form by such an LLM. The LLM may be able to output embeddings of text, images, audio, and video, and multimodal machine learning model 230 may also include one or more diffusion models for generating outputs in image, audio, and video form based on such embeddings output by the LLM. For example, multimodal machine learning model 230 may include an image diffusion model, an audio diffusion model, and a video diffusion model, each of which may generate outputs in a particular modality. Multimodal machine learning model 230 may be capable as a result of its training and/or fine tuning of automatically generating outputs in one or more modalities in response to prompts based on inputs of varying modalities. For example, multimodal machine learning model 230 can be an entry point for many use cases in a clinical setting, such as during surgery. Multimodal machine learning model 230 can utilize many specific expert models within its architecture to perform downstream tasks.

Furthermore, fine tuning algorithm 220 may be used to re-train multimodal machine learning model 230 as new training data becomes available.

FIG. 3 illustrates an example 300 of an artificial intelligence agent for machine learning based medical treatment optimization, in accordance with certain embodiments of the present disclosure.

In example 300, main AI agent 122 of FIG. 1 provides a prompt 322 along with associated data 324 (which may include data in multiple modalities) to multimodal machine learning model 230 of FIG. 2, which outputs content 326 (which may be in one or more modalities) in response. Prompt 322 may be based on a request, such as input by a medical professional via an input device, for a particular type of information or other content. The request may specify the modality for the requested content. For example, the request may be for a recommended action for handling a particular issue during a clinical procedure and for a picture illustrating the recommended action. Prompt 322 may comprise the request and may be provided to main AI agent 122 along with associated data 324 as context. Main AI agent 122 may have retrieved associated data 324 based on the request, such as based on comparing an embedding of the request to embeddings of various data items (e.g., from one or more medical devices) to determine which data items are relevant to the request (e.g., based on cosine similarity or another vector similarity comparison between the embedding of the request and the embeddings of data items). Associated data 324 may include data items determined based on such a comparison to be relevant to the request, such as having embeddings within a threshold Euclidean distance from the embedding of the request. Associated data 324 may include text, image data, video data, audio data, and/or the like.

Multimodal machine learning model 230 may analyze associated data 324 based on prompt 322 and may generate content 326 based on such analysis. For example, content 326 may include text content, image content, video content, and/or audio content that was requested in prompt 322.

FIG. 4 illustrates an example related to machine learning based medical treatment optimization, in accordance with certain embodiments of the present disclosure.

In the depicted example, a screen 400 includes a request 410 and an associated response 420. For instance, screen 400 may represent a user interface screen and/or may otherwise represent a request received via an input device (e.g., via text input, audio input, video input, touch input mouse input, and/or the like) and a response (e.g., content) provided via an output device (e.g., a screen, a speaker, and/or the like).

Request 410 includes the language “Which intraocular lens should I use?” and may have been input by a medical professional during performance of an ophthalmic procedure. Request 410 may be processed by an AI agent as described above with respect to FIGS. 1 and 3, such as in connection with associated data, and the AI agent may automatically generate (e.g., using a multimodal machine learning model) response 420 based on the request and associated data. The associated data may include, for example, patient attributes, the step or part of the ophthalmic procedure that is currently being performed, data about the patient that was captured via one or more medical devices and/or otherwise stored, live data about the patient’s current condition, live data about the medical professional’s current movements, inventory and/or stock information related to intraocular lenses within the clinical facility, information about best practices for the ophthalmic procedure, information about past ophthalmic procedures, and/or the like.

Response 420 includes a natural language response to request 410, informing the medical processional that, based on the patient’s information, one of three possible choices can be selected as an intraocular lens under the circumstances. Response 420 indicates, for each of the three possible choices, a shelf the lens can be located on and an amount of stock remaining for the lens. Response 420 also reminds the medical profession to verify the lens before implanting the lens and includes images of the three possible choices. The images 422, 424, and 426 may depict examples of the lenses and/or packaging in which the lenses can be found.

FIG. 5 illustrates another example related to machine learning based medical treatment optimization, in accordance with certain embodiments of the present disclosure.

In the depicted example, a screen 500 includes a request 510 and an associated response 520. For instance, screen 500 may represent a user interface screen and/or may otherwise represent a request received via an input device (e.g., via text input, audio input, video input, touch input mouse input, and/or the like) and a response (e.g., content) provided via an output device (e.g., a screen, a speaker, and/or the like).

Request 510 includes the language “Here are a pre-op eye image and an intra-op eye image during cataract surgery. Please combine these two images into one image and match key eye features such as limbus and blood vessel. Please output one fusion image” and may have been input by a medical professional during performance of an ophthalmic procedure. Request 510 may be processed by an AI agent as described above with respect to FIGS. 1 and 3, such as in connection with associated data, and the AI agent may automatically generate (e.g., using a multimodal machine learning model) response 520 based on the request and associated data. The associated data may include, for example, the two images indicated in request 510, information about cataract surgeries, information about eye features such as limbus and blood vessels and other key eye features, and/or the like.

Response 520 includes a natural language response to request 510, informing the medical processional the resulting fusion image is provided, and that another fusion image from a registration model is also provided. Response 520 includes image 522, which may be the requested fusion image, such as depicting a combination of the pre-op eye image and the intra-op eye image with key eye features such as limbus and blood vessels mapped to one another in the single image. Response 520 also includes image 524, which may be another fusion image from a registration model that is relevant to image 522 and/or request 510.

In another example (not depicted), a request asks for a next step in a cataract surgery and asks what equipment the staff should prepare and be ready to hand the surgeon. The response to such a request may include a natural language explanation of the next step along with an indication of which equipment should be prepared for that step (e.g., “Now is the Phaco procedure in Cataract Surgery. The next step might be View Phakic. You need to prepare the probe and Sterile Irrigation like this.”) and/or one or more images depicting the next step and/or the indicated equipment.

In another example (not depicted), a request asks for phaco tip movement and pupil dynamics to be tracked in a video of cataract surgery. The response to such a request may include one or more videos and/or images from video(s) including visual indicators of the phaco tip movement and pupil dynamics as requested.

In another example (not depicted), a request asks for a current pupil part in an image or video to be enhanced. The response to such a request may include a modified version of the image or video with the pupil part enhanced, such as using blue boost and red reflex. For example, the multimodal machine learning model may have learned based on historical data that using blue boost and red reflex produced the best enhancement to pupils in videos and may have generated the response accordingly. The response may also include a natural language explanation of how the image or video was enhanced.

In another example (not depicted), a request asks for a video of a cataract surgery to be clipped to include only the phaco tip procedure. The response to such a request may include a modified version of the video that includes only the phaco tip procedure.

These examples are included for explanation purposes, and many other examples are possible.

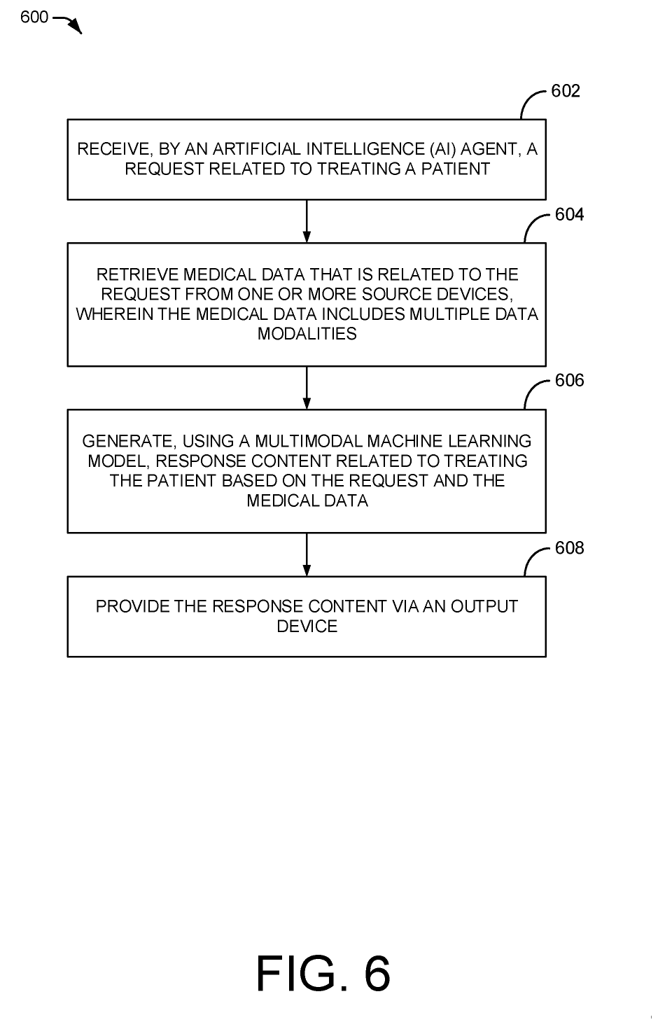

FIG. 6 illustrates an example of a process 600 related to machine learning based medical treatment optimization, in accordance with certain embodiments of the present disclosure.

In certain embodiments, the process 600 can be implemented by one or more components described above with respect to FIGS. 1-3 and/or below with respect to FIG. 6. It is noted that any number of systems, in whole or in part, can implement the process 600.

Process 600 begins at block 602, with receiving, by an artificial intelligence (AI) agent, a request related to treating a patient.

Process 600 continues at block 604, with retrieving medical data that is related to the request from one or more source devices, wherein medical data includes multiple data modalities.

Process 600 continues at block 606, with generating, using a multimodal machine learning model, response content related to treating the patient based on the request and medical data.

Process 600 continues at block 608, with providing the response content via an output device.

In some aspects, the request indicates a target data modality of the response content, and the multimodal machine learning model generates the response content according to the indicated target data modality based on the request.

In certain aspects, the multiple data modalities comprise two or more of: sensor data; text data; image data; video data; or audio data.

In some aspects, the AI agent is configured to monitor the medical data related to the patient and generate an alert of an anomaly detected using the multimodal machine learning model based on the monitoring.

In certain aspects, the medical data comprises one or more of: medical information; lab results; imaging studies; patient attributes; or medical professional activity data.

In some aspects, the one or more source devices comprise one or more of: a health monitoring device; a ventilator; a surgical instrument; an activity monitoring device; or a medical data storage device.

In certain aspects, the request and the response content relate to one or more of: surgical planning; intraoperative assistance; or postoperative care or assistance.

In some aspects, the multimodal machine learning model has been fine-tuned based on one or more of: medical information; surgery steps; surgery video annotations; surgery records; patient information; operating room inventory; or stock information.

In certain aspects, at least one of the one or more source devices comprises a local AI agent configured to analyze corresponding medical data and output inferences related to the medical data.

In some aspects, the AI agent is further configured to utilize remote cloud-based AI computing resources for generating content based on resource requirements associated with the generating of the content.

In certain aspects, the request comprises a question of which item from inventory is an appropriate item to be used based on associated medical circumstances, and wherein the response content indicates the appropriate item, a location of the appropriate item, and an image of the appropriate item.

In some aspects, the request is for a next step in a medical procedure based on associated medical circumstances, and wherein the response content indicates the next step and one or more items associated with the next step.

In certain aspects, the request is for a modification to one or more images related to a medical procedure, and the response content comprises an image generated based on the modification according to the request.

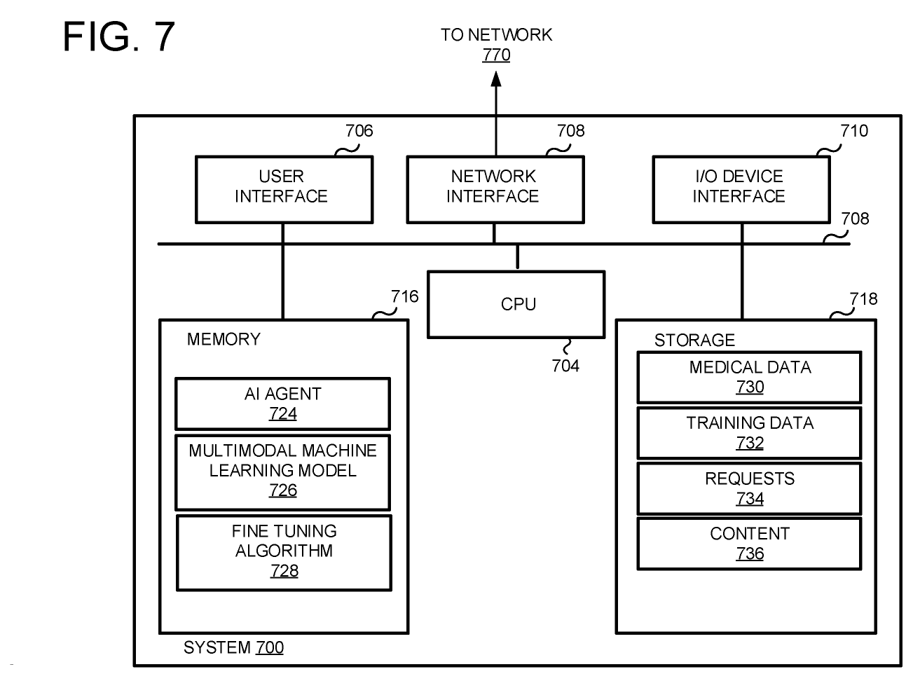

FIG. 7 illustrates an example of a system 700 for machine learning based medical treatment optimization, in accordance with certain embodiments of the present disclosure. For example, system 700 may be configured to perform method 600 of FIG. 6 and/or other aspects of the present disclosure, such as discussed above with respect to FIGS. 1-5.

As shown, system 700 includes, without limitation, central processing unit (CPU) 704, user interface 706, network interface 708, memory 716, storage 718, interconnect 708, and at least one I/O device interface 710 which may allow for the connection of various I/O devices (e.g., keyboards, displays, mouse devices, pen input, etc.) to system 700. While one or more operations are described herein as being performed by certain components of system 700, those operations may, in some embodiments, be performed by other components of system 700 and/or component(s) of other system(s). As an example, while one or more operations are described herein as being performed by CPU 705, memory 716, and/or storage 718 those operations may, in other embodiments, be performed by other components of system 700 or of a different system.

CPU 704 may be representative of one or more processing devices and/or cores. In some embodiments, CPU 704 may retrieve and execute programming instructions stored in memory 716. Similarly, CPU 704 may retrieve and store application data residing in memory 716. Interconnect 708 transmits programming instructions and application data, among CPU 704, I/O device interface 710, user interface 706, memory 716, storage 718, network interface 708, etc. In some embodiments, CPU 704 may correspond to a single CPU, multiple CPUs, or a single CPU having multiple processing cores. Additionally, in some embodiments, memory 716 represents volatile memory, such as random-access memory. In some embodiments, storage 718 may be non-volatile memory, such as a disk drive, solid state drive, or a collection of storage devices distributed across multiple storage systems.

System 700 can include a network interface 708 for connection with a data communications network (e.g., network 750), such as to communicate with other devices. The data communications network can be, or can include, one or more of a private network, a public network, a local or wide area network, the Internet, combinations of the same, and/or the like. The data communications network can include, for example, interfaces (e.g., application programming interfaces) for enabling interaction and communication between and among the components and systems of the computing environment (e.g., of FIG. 3) and/or other components and systems.

The memory 716 can include an AI agent 724, which generally represents main AI agent 122 and/or one or more of local AI agents 162, 172, and/or 182 of FIG. 1. AI agent 724 may make use of a multimodal machine learning model 726 that is also depicted in memory 716, such as to automatically generate content 736 based on requests 734. For example, multimodal machine learning model 726 may be representative of multimodal machine learning model 230 of FIGS. 2 and 3. Memory 716 further comprises a fine tuning algorithm 728, which may be representative of fine tuning algorithm 220 of FIG. 2 In other embodiments, multimodal machine learning model 726 may be trained and/or fine-tuned on a separate system from the system (e.g., system 700) on which the trained model is used to generate content.

The storage 718 can include medical data 730, which may be representative of medical data 164, 174, and/or 184 of FIG. 1. The storage 718 can also include training data 732, which may be representative of training data 210 of FIG. 2. The storage 718 can also include requests 734, which may be representative of prompt 322 of FIG. 3, request 410 of FIG. 4, and/or request 510 of FIG. 5. The storage 718 can also include content 734, which may be representative of content 326 of FIG. 3, response 420 of FIG. 4 and/or response 520 of FIG. 5.

It is noted that system 700 is included as an example, and techniques described herein may be implemented via fewer or more components, either on the same or different devices, and devices may include physical and/or virtual devices.

As used herein, a phrase referring to “at least one of” a list of items refers to any combination of those items, including single members. As an example, “at least one of: a, b, or c” or “at least one of: a, b, and c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112(f) unless the element is expressly recited using the phrase “means for” or, in the case of a method claim, the element is recited using the phrase “step for.” The word “exemplary” is used herein to mean “serving as an

example, instance, or illustration.” Any aspect described herein as “exemplary” is not necessarily to be construed as preferred or advantageous over other aspects.

What is claimed is:

1. A system for machine learning based medical treatment optimization, the system comprising:
one or more source devices configured to store or generate medical data related to a patient;
one or more interface devices configured to receive a request related to treating the patient and output response content related to treating the patient; and
an artificial intelligence (AI) agent configured to:
retrieve a subset of the medical data that is related to the request from a subset of the one or more source devices, wherein the subset of the medical data includes multiple data modalities; and
generate, using a multimodal machine learning model, the response content related to treating the patient based on the request and the subset of the medical data.

2. The system of claim 1, wherein the request indicates a target data modality of the response content, and wherein the multimodal machine learning model generates the response content according to the indicated target data modality based on the request.

3. The system of claim 1, wherein the multiple data modalities comprise two or more of:
sensor data;
text data;
image data;
video data; or
audio data.

4. The system of claim 1, wherein the AI agent is configured to monitor the medical data related to the patient and generate an alert of an anomaly detected using the multimodal machine learning model based on the monitoring.

5. The system of claim 3, wherein the medical data comprises one or more of:
medical information;
lab results;
imaging studies;
patient attributes; or
medical professional activity data.

6. The system of claim 1, wherein the one or more source devices comprise one or more of:
a health monitoring device;
a ventilator;
a surgical instrument;
an activity monitoring device; or
a medical data storage device.

7. The system of claim 1, wherein the request and the response content relate to one or more of:
surgical planning;
intraoperative assistance; or
postoperative care or assistance.

8. The system of claim 1, wherein the multimodal machine learning model has been fine-tuned based on one or more of:
medical information;
surgery steps;
surgery video annotations;
surgery records;
patient information;
operating room inventory; or
stock information.

9. The system of claim 1, wherein at least one of the one or more source devices comprises a local AI agent configured to analyze corresponding medical data and output inferences related to the medical data.

10. The system of claim 1, wherein the AI agent is further configured to utilize remote cloud-based AI computing resources for generating content based on resource requirements associated with the generating of the content.

11. The system of claim 1, wherein the request comprises a question of which item from inventory is an appropriate item to be used based on associated medical circumstances, and wherein the response content indicates the appropriate item, a location of the appropriate item, and an image of the appropriate item.

12. The system of claim 1, wherein the request is for a next step in a medical procedure based on associated medical circumstances, and wherein the response content indicates the next step and one or more items associated with the next step.

13. The system of claim 1, wherein the request is for a particular modification to one or more images related to a medical procedure, and wherein the response content comprises an image generated based on the particular modification according to the request.

14. A computer-implemented method of machine learning based medical treatment optimization, the computer-implemented method comprising:
receiving, by an artificial intelligence (AI) agent, a request related to treating a patient;
retrieving medical data that is related to the request from one or more source devices, wherein the medical data includes multiple data modalities;
generating, using a multimodal machine learning model, response content related to treating the patient based on the request and the medical data; and
providing the response content via an output device.

15. The computer-implemented method of claim 14, wherein the request indicates a target data modality of the response content, and wherein the multimodal machine learning model generates the response content according to the indicated target data modality based on the request.

16. The computer-implemented method of claim 14, wherein the multiple data modalities comprise two or more of:
sensor data;
text data;
image data;
video data; or
audio data.

17. The computer-implemented method of claim 14, wherein the AI agent is configured to monitor the medical data related to the patient and generate an alert of an anomaly detected using the multimodal machine learning model based on the monitoring.

18. The computer-implemented method of claim 17, wherein the medical data comprises one or more of:
medical information;
lab results;
imaging studies;
patient attributes; or
medical professional activity data.

19. The computer-implemented method of claim 14, wherein the one or more source devices comprise one or more of:
a health monitoring device;
a ventilator;
a surgical instrument;
an activity monitoring device; or
a medical data storage device.

20. A non-transitory computer-readable medium comprising instructions that, when executed via one or more processors of a computing system, cause the computing system to:
receive, by an artificial intelligence (AI) agent, a request related to treating a patient;
retrieve medical data that is related to the request from one or more source devices, wherein the medical data includes multiple data modalities;
generate, using a multimodal machine learning model, response content related to treating the patient based on the request and the medical data; and
provide the response content via an output device.
